(19) United States
(12) Patent Application Publication
(10) Pub. No.: US 2025/0000902 A1
(43) Pub. Date: Jan. 2, 2025

(54) METHOD FOR PRODUCTION OF BIOACTIVE SUBSTANCE USING BLOOD AND APPLICATION THEREOF

(71) Applicant: AMINOLAB CO., LTD., Sejong-si (KR)

(72) Inventors: Wha Jung CHO, Sejong-si (KR); Tae Heon HAN, Gwangmyeong-si (KR); Hae Sung PARK, Seongnamm-si (KR); Ki Woo KIM, Seoul (KR)

(21) Appl. No.: 18/705,308

(22) PCT Filed: Nov. 19, 2021
(86) PCT No.: PCT/KR2021/017072

§ 371 Date: Apr. 26, 2024

(30) Foreign Application Priority Data
Oct. 26, 2021 (KR) 10-2021-0143627

(51) Int. Cl. A61K 35/16

(52) U.S. Cl. CPC A61K 35/16; A61K 2800/805; A61K 2800/85

(57) ABSTRACT

The present invention relates to a method for production of a bioactive substance using blood, and application of the product obtained by the method, the method comprising the steps of: a) collecting animal blood treated with 0.32% trisodium citrate and centrifuging the blood to obtain plasma, b) treating the plasma with proteinase to degrade proteins therein, incubating the degraded plasma in the presence of 1-2% active carbon, centrifuging the plasma, removing the pellet, incubating the supernatant at 90-95° C. at 30 minutes or longer to inactivate enzymes and kill germs, followed by filtration through a filter to filter out bacteria, and c) spray-drying the filtrate with a spray drier to obtain a powder. The product of the present invention can be used for commercialization of protein foods, food additives, proteinous cosmetic materials, and/or various hair-related cosmetic products such as a functional shampoo for alleviating hair loss.

### TECHNICAL FIELD

The present disclosure relates to a method for production of a bioactive substance using blood and an application thereof.

### BACKGROUND ART

Recently, human average lifespan has been increasing due to improvements in living standards and advances in modern medicine, and as a result, interest in health and the desire for youth and beauty are increasing. This atmosphere extends to the medical and dietary fields beyond the beauty field, where various beauty technologies, medicine, and foods are being developed, and a lot of research in related fields is also underway.

Unlike artificially synthesized cosmetics, natural cosmetic materials refer to materials that utilize components naturally produced by living things, and interest in natural cosmetic materials is increasing due to controversy over the harmfulness of chemical and drug ingredients and marketing needs.

Recently, various plant extracts or herbal extracts are preferred and widespread, but their efficacy and components are often unclear, and despite the controversy over their efficacy, there is still a lot of room to discover materials with new efficacy from natural products.

When it is a safe component with excellent efficacy, there is no need to distinguish between animals and plants, and it is meaningful in expanding consumer choice.

Therefore, it is more public interest and necessary to develop technology to discover natural products that are easy to obtain and cost-effective rather than rare and expensive materials.

### PATENT DOCUMENT IN RELATED ART

Korean Patent Publication No. 10-2013-0029294

### DISCLOSURE

### Technical Problem

The present disclosure was created in response to the above-described necessity, and the present disclosure is for the purpose of providing a method for production of a high value-added material from animal blood.

The present disclosure is also for the purpose of providing a high value-added materials from animal blood.

### Technical Solution

In order to achieve the above purpose, the present disclosure provides a method for a production of a bioactive substance using blood, including:
a) collecting animal blood treated with 0.32% trisodium citrate and centrifuging the blood to obtain plasma;
b) treating the plasma with proteinase to degrade proteins therein, incubating the degraded plasma in the presence of 1-2% active carbon, centrifuging the plasma, removing the pellet, incubating the supernatant at 90-95° C. at 30 minutes or longer to inactivate enzymes and kill germs, followed by filtration through a filter to filter out bacteria, and
c) spray-drying the filtrate with a spray drier to obtain a powder.

In one embodiment of the present disclosure, the anticoagulant is preferably trisodium citrate, but is not limited thereto.

In another embodiment of the present disclosure, the protein-degrading enzyme is preferably subtilisin, but is not limited thereto.

In still another embodiment of the present disclosure, the protein-degrading enzyme is preferably added to the plasma in an amount of 0.7% or more, more preferably 1% (w/v), but is not limited thereto.

In yet another embodiment of the present disclosure, in the method, when plasma is separated without hemolysis of red blood cells, activated carbon is not treated, but is not limited thereto.

In addition, the present disclosure provides a product produced by the method of the present disclosure.

In addition, the present disclosure provides a food or food additive composition including the product of the present disclosure as an active ingredient.

In addition, the present disclosure provides cosmetic composition including the product of the present disclosure as an active ingredient.

In one embodiment of the present disclosure, the cosmetic composition is anti-aging cosmetics, hair and scalp cosmetics, massage cosmetics, mask packs, skin cosmetics, solar skin protection cosmetics, and hair shampoos, but is not limited thereto.

The cosmetic composition of the present disclosure may be formulated with one or more materials selected from the group consisting of shampoo, softening tonic, nutritional tonic, moisture cream, nutritional cream, massage cream, essence, ampoule, gel, eye cream, cleansing cream, cleansing foam, cleansing water, pack, spray, and powder.

The cosmetic composition may be formulated by methods in the related art. In formulating skin external preparations, reference may be made to information disclosed in Remington's Pharmaceutical Science, Mack Publishing Company, Easton PA, and in formulating cosmetic compositions, and reference may be made to International cosmetic ingredient dictionary, 6th ed (The cosmetic, Toiletry and Fragrance Association, Inc., Washington, 1995).

Specifically, the cosmetic composition can be prepared in a general emulsified formulation and solubilized formulation. The composition may be formulated as, for example, toners such as softening tonic or nutritional tonic; emulsions such as facial lotion and body lotion; creams such as nutritional cream, moisture cream, eye cream and the like; essence; cosmetic ointment; spray; gel; pack; sunscreen; makeup base; foundations such as liquid type, solid type, spray type and the like, but is not limited thereto. In addition, the skin external preparation may be formulated as an ointment, patch, gel, cream, or a spraying agent, but is not limited thereto.

In each formulation, the cosmetic composition may be appropriately mixed with other components in addition to the above essential components within a range that does not impair the purpose according to the present disclosure, depending on the type of formulation or purpose of use.

The cosmetic composition may include a generally acceptable carrier and may be appropriately mixed with, for example, oil, water, surfactant, moisturizer, lower alcohol, thickener, chelating agent, colorant, preservative, fragrance or the like, but is not limited thereto.

The acceptable carrier may vary depending on the formulation. When formulated as ointments, pastes, creams or gels, for example, animal oils, vegetable oils, wax, paraffin, starch, tragacanth, cellulose derivatives, polyethylene glycol, silicone, bentonite, silica, talc, zinc oxide, or mixtures thereof may be used as a carrier component.

When the cosmetic composition is formulated as a powder or spray, lactose, talc, silica, aluminum hydroxide, calcium silicate, a polyamide powder, or mixtures thereof as a carrier component may be used, and in the case of sprays, a propellant such as chlorofluorohydrocarbon, propane, butane or dimethyl ether may be used.

When the cosmetic composition is formulated as a solution or emulsion, as a carrier component, a solvent, a solubilizing agent, or an emulsifying agent may be used, for example, water, ethanol, isopropanol, ethyl carbonate, ethyl acetate, benzyl benzoate, propylene glycol, or 1,3-butyl glycol oil may be used, and in particular, a cottonseed oil, a peanut oil, a corn germ oil, an olive oil, a castor oil, sesame oil, fatty acid esters of glycerol, polyethylene glycol, or sorbitan may be used.

When the cosmetic composition of the present disclosure is formulated as a suspension, a liquid diluent such as water, ethanol or propylene glycol, an ethoxylated isostearyl alcohol, a suspending agent such as polyoxyethylene sorbitol ester and polyoxyethylene sorbitan ester, microcrystalline cellulose, aluminum metahydroxide, bentonite, agar, tragacanth, or the like may be used as a carrier component.

In addition, the cosmetic composition may further contain fatty substances, organic solvents, solubilizing agents, thickening agents, gelling agents, softening agents, antioxidants, suspending agents, stabilizers, foaming agents, fragrances, surfactants, water, ionic or nonionic emulsifiers, fillers, sequestering and chelating agents, preservatives, blocking agents, wetting agents, essential oils, dyes, pigments, hydrophilic or lipophilic active agents commonly used in the industry depending on the quality or function of the final product, or auxiliaries commonly used in the field of cosmetology or dermatology, such as any other ingredients commonly used in cosmetics. The auxiliaries and their mixing ratio may be appropriately selected so as not to affect the desirable properties of the cosmetic composition according to the present disclosure.

In addition, the present disclosure provides medium composition including the product of the present disclosure as an active ingredient.

Hereinafter, the present disclosure will be described.

Slaughter blood is a highly valuable life resource as it is rich in various proteins and minerals, and an average protein content in the blood is around 18-20%, a various mineral content is 1-2%, and the rest is mostly water.

An animal blood protein is ‘complete protein’ in which about 20 types of amino acids are evenly distributed, pig blood has been used for food for a long time and is ‘food’ that is safe when handled hygienically, and slaughter blood, a by-product of slaughter, is classified as ‘food’ under the Food Act.

In Europe, pig blood is used to produce medicines and food additives such as albumin.

In addition, animal raw materials such as silkworm cocoons, snails, placenta, horse oil, eel, earthworm, and deer oil are used in all cosmetics, and animal proteins are superior to vegetable proteins in terms of amino acid content, distribution, and type.

It is difficult to use human blood, but pig blood can be used, and it is hygienic and easy to source high-quality raw materials; ‘Placenta’, which is widely used as a substitute for human placenta among cosmetic ingredients, is a safe material made by hydrolyzing pig placenta.

The present disclosure focuses on the high utility value of highly nutritious life resources that have been abandoned and provides a method for production of high value-added materials from animal blood, in which the animal blood may be converted into high-value-added materials such as functional cosmetics materials and food materials by breaking away from low-value-added resources such as fertilizers and feeds, the animal blood is a complete protein resource that contains over 20 types of amino acids from animal sources and essential and non-essential amino acids, and rich in various growth factors and minerals in addition to protein.

Since the recent enforcement of the London Convention completely banned the dumping of land-based waste into the ocean, land-based waste is on the rise again, and in line with the trend of strengthening global eco-friendly policies and environmental regulations, there is an urgent need to reduce waste through the recycling of slaughter blood, and accordingly, the method of the present disclosure has the effect of relieving the economic burden on slaughterhouses and reducing environmental pollution by reducing processing costs due to waste reduction.

Currently, slaughter blood is limited to producing simple liquid fertilizer or blood meal feed, but because blood (whole blood) contains a large amount of various proteins and useful components, not only does slaughter blood has a wide range of uses, but it also has excellent utility value as plasma contains albumin, immune proteins, various peptides, and free amino acids.

Therefore, it is necessary to develop high-value use methods (materials) rather than simple recycling of these highly nutritious life resources (fertilizer, blood meal, etc.).

The characteristics of the preparation process of the present disclosure are as follows.

### Preventing Blood Coagulation Through Anticoagulant Pretreatment

When blood flows out of the blood vessel, platelets are immediately activated to prevent continued loss, and a blood coagulation reaction occurs through interaction with coagulation factors. Therefore, to prevent blood coagulation, it is treated with 0.32% trisodium citrate to ensure uncoagulated blood.

### Plasma Separation Using Continuous Centrifuge

Blood is composed of blood cells and plasma components, and each component may be easily separated using a general centrifuge in the laboratory. A plasma fraction is obtained by centrifuging at 3200 rpm for 25 minutes using a research centrifuge.

In addition, as continuous separation of plasma and blood cells is required to produce industrial materials, an industrial continuous centrifuge may be used. Among various types of centrifuges, tubular type or disk type centrifuges capable of three-phase separation are suitable, but are not limited to thereto.

### Protein Hydrolysis Using Enzyme Digestion Method

Proteins have a primary structure in which amino acids are combined through peptide bonds, and secondary and tertiary structures in many non-covalent bonds. Even proteins that perform the same function in each species have different structures, and this has the potential to cause individual-specific allergic reactions. Therefore, in order to prevent allergic reactions and improve absorption and increase efficacy by hydrolyzing proteins with enzymes in advance, the separated plasma is treated with an appropriate amount of protein-degrading enzymes and reacted at 50-55° C. for 12 hours or more.

### Purification of Plasma-Hydrolyzed Extract Through Centrifugation and Filtration Process

Plasma is a fraction from which red blood cells have been removed and does not have the characteristic red color and smell of blood. However, during hydrolysis, hydrophobic components dissolved together with the protein in plasma are eluted and precipitated in the aqueous solution as the protein is decomposed. This is first removed by centrifugation and then filtered through a filter.

When separating plasma using industrial centrifuges, some red blood cells may be lysed. When reddish plasma is used, the plasma is decolorized using an appropriate amount of activated carbon before centrifugation.

### Production of Plasma-Hydrolyzed Extract Powder Material

Plasma-hydrolyzed extract is vulnerable to microbial growth due to its high concentration of nutrients, so preservative treatment is inevitable during storage. In addition, there is a possibility that the concentration of the produced extract may change depending on the condition of the raw materials, which makes quantitative use of the active ingredient difficult. To overcome these matters, the hydrolyzed extract is sterilized at 90° C. for 30 minutes or sanitized using a sterilizing filter, and then moisture is removed and powdered through spray-drying or freeze drying.

The present disclosure has the following differences compared to existing methods.

### Elimination of Liquefaction (Pulverization) Process:

Since the existing method requires collecting blood as is, the coagulated blood had to be liquefied with a powerful pulverizing blade immediately upon receipt, which causes a large amount of foam to be generated, freshness is threatened due to unnecessary heat contact with the blood during the pulverzing process, and fine pulverizing of hairs in the blood, and there are difficulties with filtration in metal mesh filtering, and the generation of loud noise in the workplace is inevitable due to the adoption of large motors to shorten time.

The present disclosure has advantages in terms of preventing coagulation by treating blood with an anticoagulant when collecting blood, slimming the process for blood that requires rapid processing, saving time and energy, and maintaining freshness compared to existing methods.

### Centrifugation Process:

Since the existing method recycled whole blood as is, there are technical limitations in recycling various fields such as plasma and heme iron. Blood cells place a huge burden on filtration. Unlike whole blood, separated plasma is yellow and has very little unusual odor, making it easy to filter, whereas whole blood, which also includes blood cells, is black after decomposition and has a strong unusual odor, making decolorization and deodorization a major burden, and due to the powerful filtration process, the cost and time as well as the loss of active ingredients were considerable, reaching 30-40%.

The present disclosure can be expected to be recycled in various fields while significantly reducing the filtration burden through the introduction of a centrifugal separation process and minimizing the loss of active ingredients and has the effect of diversifying the fields of use of plasma and blood cells, improving blood recycling value, and increasing purity by completely removing impurities.

### Unification of Filtration Process:

Previously, activated carbon (3-5%) was input directly to the solution in which whole blood was decomposed, and then filtration was performed using coated diatomaceous earth, filtration once is not enough, filtration needs to be performed at least twice through the same process, which causes many inconveniences, such as concerns about contamination due to repeated manual work of washing and re-coating diatomaceous earth after the first filtration, and disposal of activated carbon and diatomaceous earth used in filtration as waste.

The present disclosure eliminates the need for activated carbon through three-stage filtering or significantly reduces the amount used before, and as a result, secondary waste is not generated or reduced, and time and labor are reduced by simplifying the process.

### Spray Drying:

In the previous process, dehydration needs to be performed before drying to reduce the drying burden. At this time, since a water content was about 70% and it was difficult to separately recycle the dehydrated liquid, it had to be treated as wastewater. Spray drying does not require a separate dehydration process, may significantly reduce the drying burden due to concentration, and has a sterilization process before drying. In addition, since a separate dehydration process is hygienically unnecessary compared to previous processes, such as high heat treatment of 100° C. or more during drying and packaging immediately after drying spray-drying minimizes the generation of wastewater, does not require re-pulverizing of unevenly sized particles, and reduces the risk of bacterial contamination during the process due to high-temperature drying and packaging immediately after drying.

### Advantageous Effects

The present disclosure disseminates the production and technology of high value-added materials using slaughter blood, a highly nutritious life resource that is discarded, can expand the horizons of domestic blood resources by moving away from fertilizer and feed resources and turning them into high value-added materials, has the effect of replacing imports of amino acids used in protein foods, food additives, protein cosmetic materials, and the like, can be developed as a raw material for various derivative products such as health supplements and beverages to expand the range of use of natural amino acids, and can be used to commercialize various hair-related cosmetics, such as functional shampoos for hair loss relief.

### BRIEF DESCRIPTION OF DRAWINGS

FIG. 1 is a diagram comparing the process of the present disclosure with the existing method.

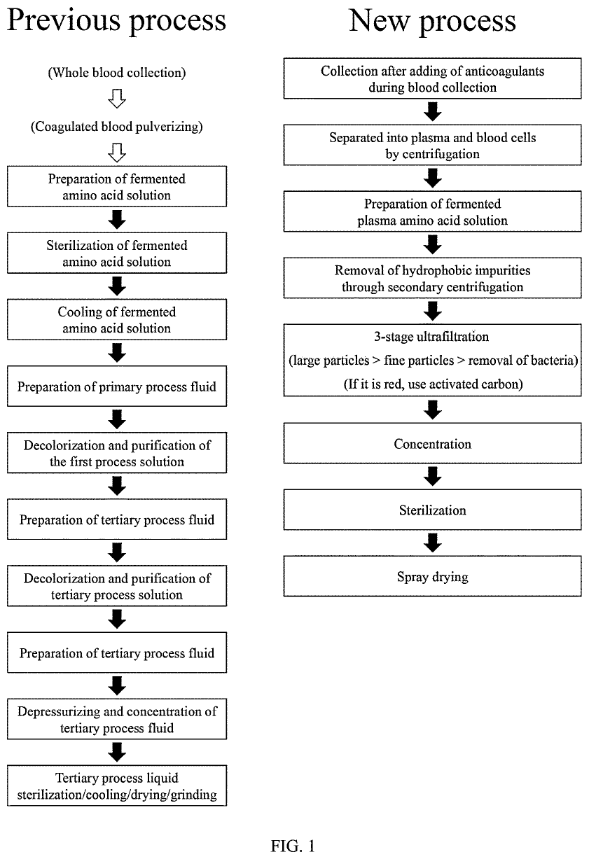

FIG. 2 is a preparation process chart of a bioactive substance using plasma of the present disclosure.

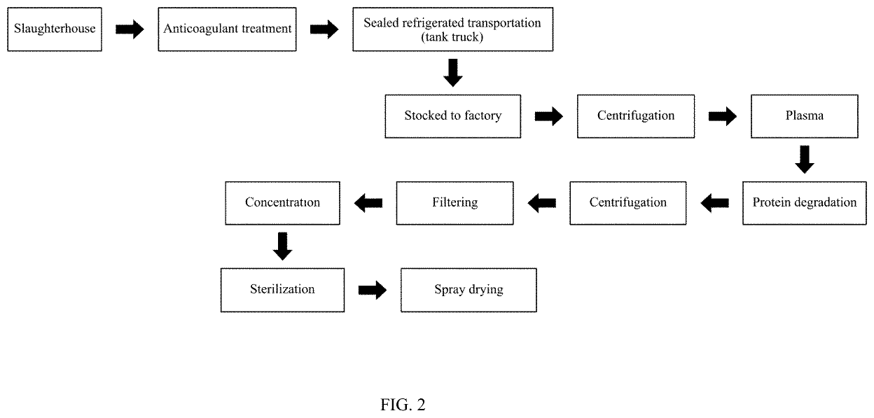

FIG. 3 shows blood treated with 0.32% trisodium citrate, plated in equal amounts, and centrifuged at 3000 g for 25 minutes at room temperature with break 0 conditions.

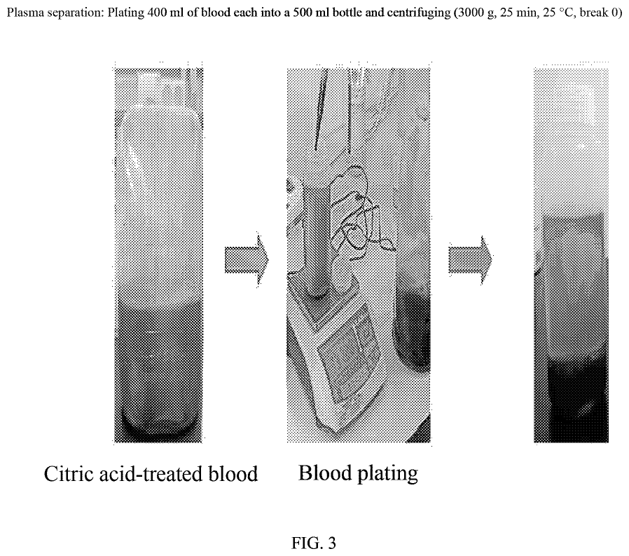

FIG. 4 shows the results of treating plasma with enzymes according to concentration, placing them in a shaking incubator, reacting at 55° C., observing color changes over time, and confirming the degree of protein decomposition through Sodium Dodecyl Sulphate-Polyacrylamide Gel Electrophoresis (SDS-PAGE).

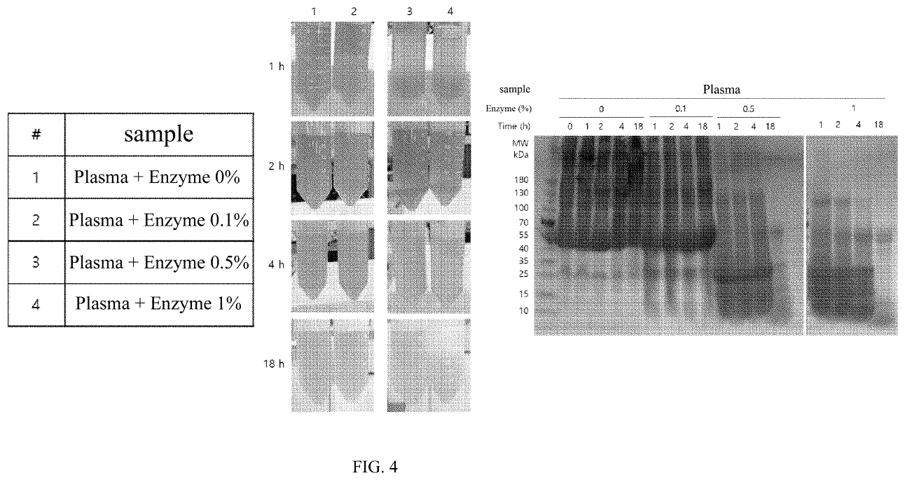

FIG. 5 shows the plasma with precipitate after protein decomposition centrifuged at 3000 g for 25 minutes.

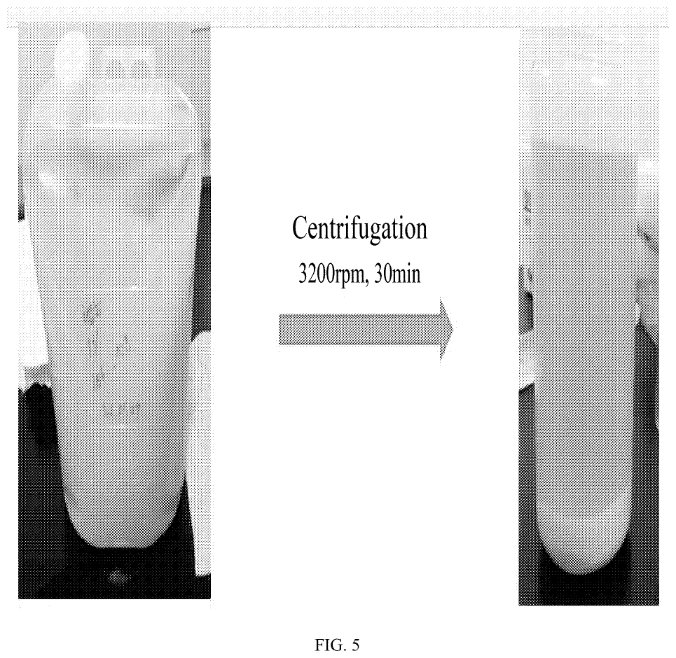

FIG. 6 shows plasma and final spray-dried powder that were first removed by centrifugation to remove impurities and then subjected to precipitation filtering and activated carbon filtering.

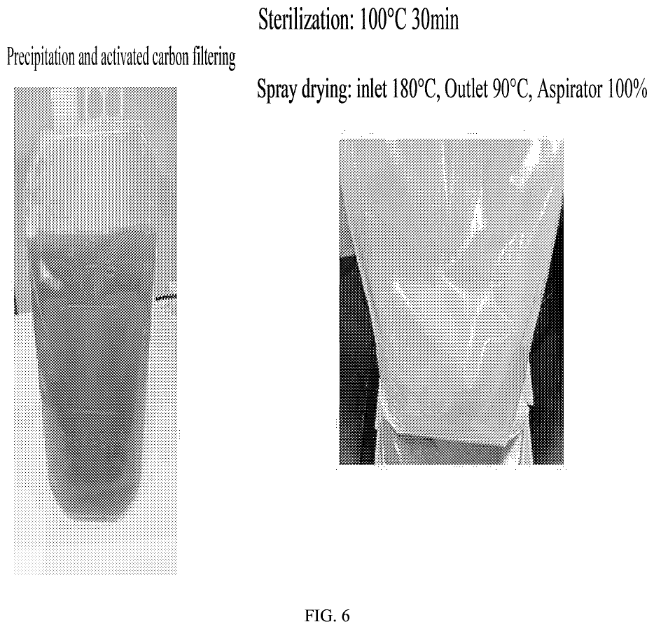

FIG. 7 shows plasma separated by a continuous centrifuge and plasma after protein decomposition.

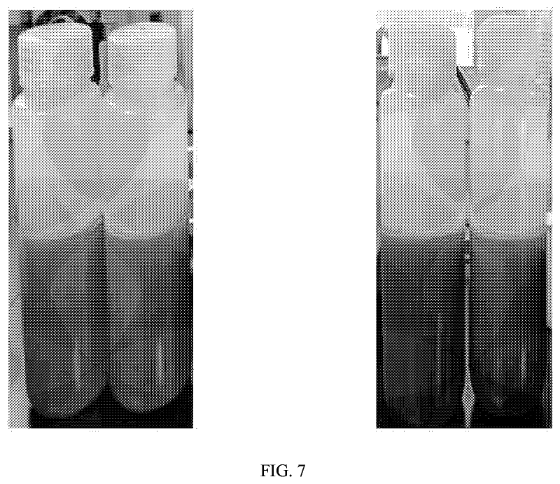

FIG. 8 is a diagram comparing the degree of decolorization by treating activated carbon at different concentrations.

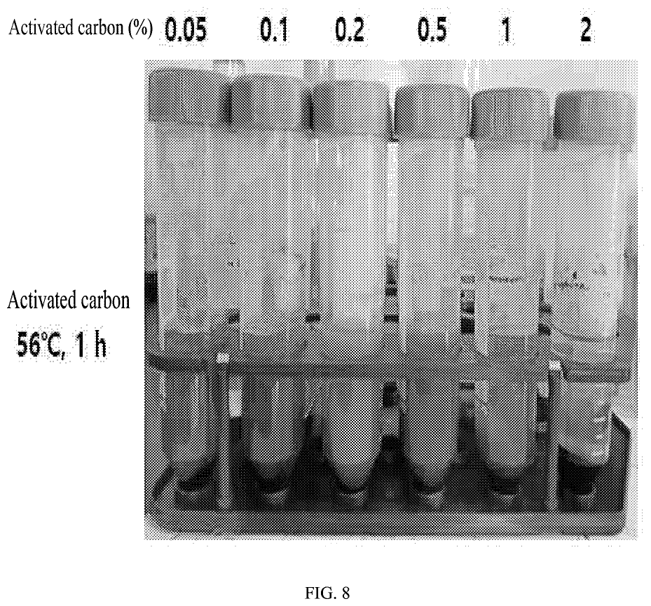

FIG. 9 shows plasma decolorized by treatment with 2% activated carbon and lyophilized powder.

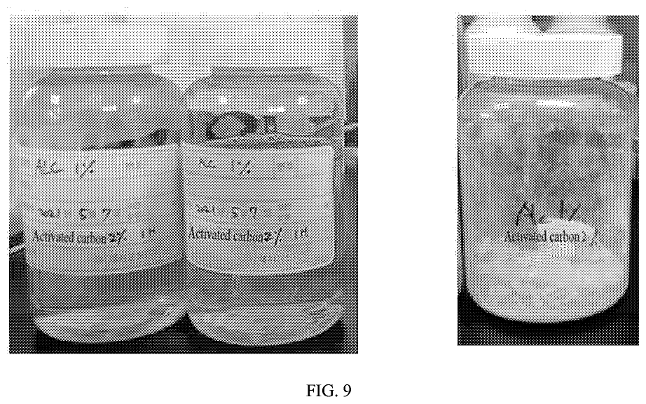

FIG. 10 is a graph comparing the protein molecular weight distribution of undigested plasma and enzyme-digested plasma using HPLC.

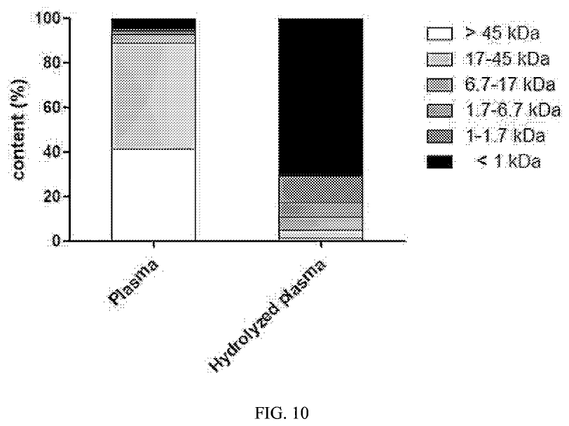

### MODES OF THE INVENTION

The present disclosure will be described in more detail below through non-limiting examples. However, the following examples are intended to illustrate the present disclosure, and the scope of the present disclosure is not to be construed as limited by the following examples.

### Examples: Preparation Process Chart of Bioactive Substance Using Plasma

400 liters of blood treated with 0.32% trisodium citrate is delivered to the factory by tank lorry from the slaughterhouse, and centrifuged under the condition of RPM of 8,000-9000, an injection speed of 1,200-1,500 ml/min, a discharge of 100-300 s, and a pressure of 2-3 bar using Alfa Laval's Clara 20-obtain about 200 L of plasma.

Plasma was treated with 1% endopeptidase (subtilisin; trademark Alcalase®) to decompose proteins for 14-18 hours at 50-56° C., 1-2% activated carbon was added to the decomposed plasma to incubate at 50-56° C. for 1-3 hours, centrifuged under the condition of 8,000-9,000 rpm, an injection speed of 1,200-1,700 ml/min, a discharge of 200-500 s, and a pressure 2-3 bar to remove precipitates, incubated at 90-95° C. for 30 minutes or more to be inactivated with enzymes and sterilized, and then sequentially filtered through 1 μm, 0.45 μm, and 0.2 μm filters to remove bacteria.

About 150 liters of the filtrate was spray-dried using a large spray dryer (EIN SYSTEM CO., LTD) of the Chuncheon Bio Industry Promotion Agency under the conditions of inlet 180-190° C., outlet 90-100° C., and feed flow 25-32% to obtain about 15 kg of powder.

### Example 2: Comparison of Plasma Protein Decomposition Rates According to Enzyme Concentration

When trisodium citrate was used as an anticoagulant, it was used in various amounts ranging from 0.32% to 0.6%. When testing blood, tubes coated with 0.38% are mainly used, and when used as a solution, a 4% solution was mixed with blood in a ratio of 1:9 to make a final 0.4%.

In preliminary experiments, it was confirmed that blood coagulation did not occur when 0.32% was used in pig blood, so it is commercially preferable to use 0.32% trisodium citrate in the production of this plasma material.

Blood treated with 0.32% trisodium citrate was plated in equal amounts, centrifuged at 3,000-3,200 rpm for 25 minutes at room temperature to separate plasma, treated with enzymes by concentration, and reacted at 50-56° C. in a shaking incubator, and color changes were observed over time, and the degree of protein degradation was confirmed through gradient SDS-PAGE.

As a result, it was confirmed that when 0.5% or more of the enzyme was treated for 18 hours or more, the protein band of 100 kDa or more was significantly reduced, and when the enzyme was 1%, the protein band decreased rapidly after 4 hours. When the enzyme concentration was further refined and re-tested, 100 kDa or more of the band decreased significantly when reacted at 0.7% for 14 hours or more, showing a concentration-dependent trend, and it was confirmed that the highest decomposition rate was achieved when treated with 1% enzyme.

The recommended concentration of AL-zyme is 1-3% of the protein amount, and theoretically, 0.08 to 0.24% is appropriate when decomposing plasma proteins (approximately 8%), but experimental results show that treatment with 0.7% or more is required.

### Example 3: Protein Molecular Weight Distribution Analysis Using HPLC

In a preliminary experiment, the protein-degrading activity was compared by treatment with Protamex, an endopeptidase such as Alcalase, and it was confirmed that the activity of Alcalase was about 10 times superior, so it is appropriate to use Alcalase in the production of this plasma material.

Plasma was treated with 1% or 1.2% enzyme to decompose proteins for 14 hours or more at 50-56° C. and remove precipitates, and the molecular weight distribution of the peptide was analyzed using HPLC (Alliance e2695, Waters) at the Korea Basic Science Institute Seoul Center.

As a result, it was confirmed that there was no difference in the content of small peptides of 0.9 kDa or less between the 1% (76.2%) and 1.2% (75.3%) treated samples. Therefore, it is appropriate to use 1% of enzyme.

### Example 4: Production of Enzymatically Decomposed Plasma Powder at Lab Scale

It was confirmed that when plasma, a yellow transparent liquid, was treated with enzymes to decompose proteins, an insoluble precipitate was formed. This was first removed by spinning down for 20-30 minutes at 2,800-3,200 rpm using a laboratory centrifuge, and then subjected to secondary removal using a precipitation filter for a water purifier and an activated carbon filter. This was spray dried using a small laboratory spray dryer under the conditions of inlet 180-200° C., outlet 90-100° C., and feed flow 15-25%.

During spray-drying, when the inlet or outlet temperature is too high, the protein powder will stick and carbonize, and when the outlet temperature is low, drying will not work properly. Spray drying conditions need to be changed depending on the type of the spray dryer, solution components, viscosity, and the like, and since the particle size and degree of drying vary depending on the size of the device, it cannot be limited to these conditions, but in the case of this enzymatically decomposed plasma material, the conditions when using a small spray dryer were inlet 180-200° C., outlet 90-100° C., and feed flow 15-25%, the conditions when using medium spray dryer were inlet 180-200° C., outlet 90-100° C., and feed flow 25-40%, and the conditions when using large-scale spray dryer were the same as those of Example 1.

### Example 5: Comparison of Plasma Decomposition and Activated Carbon Concentration Separated Using Continuous Centrifuge

100 L of blood treated with 0.32% trisodium citrate was centrifuged using Clara 20 under the condition of RPM of 8,000-9,000, an injection speed of 1,200-1,500 ml/min, discharge of 100-300 s, and a pressure of 2-3 bar, equal amounts of obtained plasma were plated, treated with 1% Alcalase, and proteins were decomposed for 14 hours or more under the conditions of 50-56° C. and 230-270 rpm using a shaking incubator.

The decomposed plasma was plated in 15 ml portions into 50 ml tubes, treated with 1, 2, 4, and 8% powdered activated carbon, and decolorized and deodorized in a shaking incubator under the conditions of 50-56° C. and 230-270 rpm for 1 hour, spun down for 30 minutes at 2,800-3,200 rpm using a laboratory centrifuge to remove activated carbon and impurities, and finally filtered through a 0.2 μm filter.

As a result of measuring the solid concentration of each solution and comparing the color and odor, only 2% treatment had a significant decolorization effect, the unusual odor remaining during 1% treatment was completely removed, and the solid concentration was also high.

It was confirmed that the best decolorization effect was achieved when using 8% activated carbon, but because too much organic matter in the plasma was removed by the high concentration of activated carbon, treatment of 1-2% was most appropriate for producing plasma materials. It was preferable that when plasma was separated without hemolysis of red blood cells, activated carbon was not treated.

1. A method for production of a bioactive substance using blood, comprising:
a) collecting animal blood treated with 0.32% trisodium citrate and centrifuging the blood to obtain plasma;
b) treating the plasma with proteinase to degrade proteins therein, incubating the degraded plasma in the presence of 1-2% active carbon, centrifuging the plasma, removing the pellet, incubating the supernatant at 90-95° C. at 30 minutes or longer to inactivate enzymes and kill germs, followed by filtration through a filter to filter out bacteria, and
c) spray-drying the filtrate with a spray drier to obtain a powder.

2. The method of claim 1, wherein the anticoagulant is trisodium citrate.

3. The method of claim 1, wherein the protein-degrading enzyme is subtilisin.

4. The method of claim 1, wherein the protein-degrading enzyme is added at 1% (w/v) to the plasma.

5. The method of claim 1, wherein when plasma is separated without hemolysis of red blood cells, activated carbon is not treated.

6. A product produced by the method of claim 1.

7. A food or food additive composition comprising the product of claim 6 as an active ingredient.

8. A cosmetic composition comprising the product of claim 6 as an active ingredient.

9. The cosmetic composition of claim 8, wherein the cosmetic composition is anti-aging cosmetics, hair and scalp cosmetics, massage cosmetics, mask packs, skin cosmetics, solar skin protection cosmetics, and hair shampoos.

10. A medium composition comprising the product of claim 6 as an active ingredient.
